(19) United States
(12) Patent Application Publication
(10) Pub. No.: US 2017/0119066 A1
(43) Pub. Date: May 4, 2017

(54) Women's shorts and pants equipped with access flap for urination

(71) Applicant: Laurette Marie Robinson, Anthem, AZ (US)

(72) Inventor: Laurette Marie Robinson, Anthem, AZ (US)

(21) Appl. No.: 14/999,723

(22) Filed: Jun. 20, 2016

Related U.S. Application Data
(60) Provisional application No. 62/285,511, filed on Nov. 2, 2015.

(51) Int. Cl. A41D 1/06; A41D 27/20; A61F 13/74; A41D 1/22

(52) U.S. Cl. CPC A41D 1/065; A41D 1/22; A41D 27/20; A61F 13/74; A41D 2300/32; A41D 2300/324; A41D 2300/30

(57) ABSTRACT

The current invention consists of women's pants or shorts that are equipped with an access flap when retracted creates an opening in the crotch area enabling the user to urinate without the need of pulling their pants down. The shorts are fashioned with or without a permanent or detachable inner liner which takes the place of underwear. The inner liner has a permanent opening over the urethra in order to allow urine to flow without being impeded by clothing. The outer layer, the actual shorts themselves, have a releasable fastened flap that is opened by pulling preferably from back to front exposing the underlying liner in the area of the opening over the urethra. A woman can freely straddle her legs apart and urinate without any clothing getting saturated.

### CROSS-REFERENCE TO RELATED APPLICATIONS

Provisional patent application No. 62/285.511

### BACKGROUND OF THE INVENTION

The disclosure is related to the clothing and apparel industry. When women are in the outdoors enjoying nature, or otherwise do not have access to restroom facilities, they often find themselves needing to urinate. This can be a predicament for them because they are unable to find a private place to relieve themselves. If they are able to find a private place, they find themselves in a very vulnerable situation due to their need to pull their shorts or pants down. Many women will painfully hold their bladder for hours in order to avoid the embarrassing need to expose themselves. Oftentimes women find themselves urinating on their shorts due to the awkward and cumbersome squatting position required in order to empty their bladder. The present invention is a viable solution for this problem, A woman can now have at least as much convenience during urination as a man. This is accomplished by a releasable flap that opens the area were urination occurs and allows for urination to be accomplished through an opening in the crotch area of pants and shorts. This subject matter could not be found in the prior art.

### BRIEF DESCRIPTION OF THE DRAWINGS

FIG. 1 from a woman's frontal view, depicts an access flap installed into the crotch of women's shorts for convenient urination. The flap, in the open position, exposes the urethra which gives way for a stream of urine to flow free from obstructions without the need to remove the pants.

FIG. 2 the access flap shown in FIG. 1 is in the closed position.

### DETAILED DESCRIPTION OF THE INVENTION

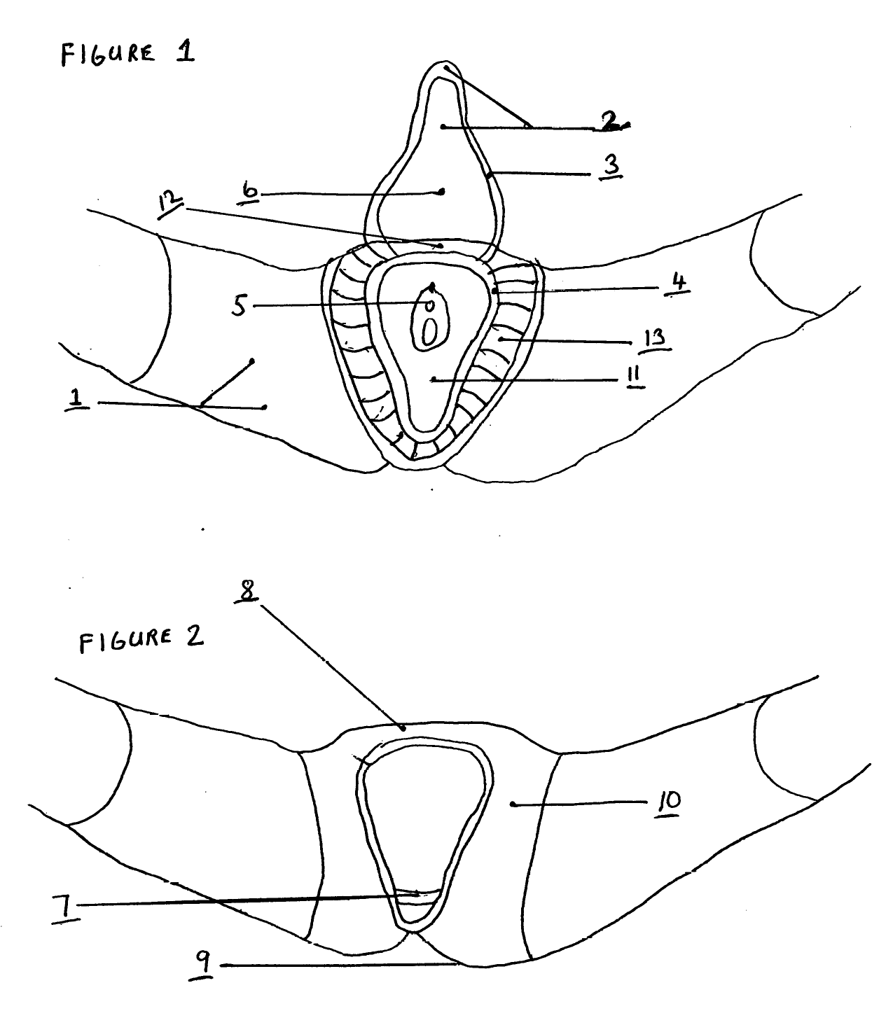

Women's shorts/pants 1 are equipped with an opening 11 in the crotch area for convenient urination. The opening is covered by an access flap 2. When access flap 2 is in the open position the urethra 5 is exposed which gives way for a stream of urine to flow free from obstructions. The access flap 2 is shown in the open position in FIG. 1 and is in the closed position in FIG. 2 . The access flap 2 is preferably opened from the rear side 9 and then pulled towards the front side 8 . The periphery of the access flap 2 along with the corresponding periphery of the opening 11 are preferably fabricated with Velcro 3 and 4. The hook side of the Velcro 4 faces away from the body 4. The soft and gentle loop side 3 faces the body to avoid irritation. In case the access flap 2 fits tightly in it's pocket, 4+11 an elastic strap 7 can be incorporated into the access flap 2 to assist in opening the access flap opening 11+4. The material that covers the inner surface of the access flap 2 is also soft and gentle. In fact, the entire inner surface of access flap can be a solid piece of Velcro soft and gentle loop material. The flap is preferably and permanently attached to the shorts at the front edge 12 of the access flap

In one embodiment, a thicker material pad 13 with an opening that corresponds to the opening 4+11 can be installed into the shorts/pants in order to create a recessed pocket so that the access flap 2 will seat seamlessly into the recessed pocket 4+11.

In another embodiment, an inner liner 10 can be fabricated adjacent to the access flap opening 4+11. The liner10 would be fabricated with an opening 11 corresponding to access flap opening 11.

1. Female pants and shorts equipped with a urination Access flap which releasably opens and exposes the urethra area in order to assist a woman in urination

2. The pants and shorts mentioned in claim one are equipped with an inner liner.

3. The pants and shorts in claim one are equipped with a liner that has an access opening in the area of the urethra.

4. The flap covering the access opening mentioned in claim one is fastened with Velcro.

5. The flap covering the access opening mentioned in claim one is fastened with snaps.

6. The flap covering the access opening mentioned in claim one is fastened with magnets.

7. The flap covering the access opening mentioned in claim one is preferably attached or sewn to the shorts/pants at it's anterior margin.

8. The access opening mentioned in claim one is surrounded by a sewn in pad creating a recessed pocket which allows the access flap to seat more seamlessly.

9. The flap covering the access opening mentioned in claim one is soft and absorbent on the underside.

10. The underside of the flap covering the access opening mentioned in claim one is equipped with a pocket able to receive a feminine hygiene absorbable pad.

11. An undergarment covering the lower portion of a woman's private area with an opening exposing the urethra.
